(19) United States
(12) Patent Application Publication
(10) Pub. No.: US 2025/0381342 A1
(43) Pub. Date: Dec. 18, 2025

(54) STREAMLINING MANAGEMENT OF MULTIPLE INFUSION SETS USING RADIO-FREQUENCY IDENTIFICATION DEVICES

(71) Applicant: CareFusion 303, Inc., San Diego, CA (US)

(72) Inventors: Adel SHAMS, Verona, NJ (US); Shirley RICHARD, Vernon Hills, IL (US); Deborah Perdita FRONZA, Trento (IT); Juliette ROY, Geneva (CH)

(21) Appl. No.: 18/747,247

(22) Filed: Jun. 18, 2024

(51) Int. Cl. A61M 5/168

(52) U.S. Cl. CPC A61M 5/16877; A61M 2205/3553; A61M 2205/3561; A61M 2205/502; A61M 2205/581; A61M 2205/582; A61M 2205/583; A61M 2205/6009

(57) ABSTRACT

An example system for streamlining management of multiple infusion sets includes first and second infusion sets. The first and second infusion sets each include respective infusion lines and respective UHF RFID tags affixed to the respective infusion lines. Each of the UHF RFID tags is configured to receive information associated with the respective infusion sets, store the information, and transmit the information to an RFID reader. The RFID reader is configured to transmit the information to one or more servers after receiving it from the UHF RFID tags. Optionally, the system may also include locator devices affixed to each of the first and second infusion sets. The locator devices are each configured to receive an identification request and, responsive to receiving a request, illuminate an LED, activate a speaker, or activate a vibrator.

### TECHNICAL FIELD

The present disclosure relates, generally, to infusion therapy technologies and, more specifically, to improving infusion therapies that involve several simultaneous infusions.

### BACKGROUND

In the context of infusion therapy, “spaghetti syndrome” refers to the tangling of infusion lines connected to a single patient. This “syndrome” is all too common in intensive care units and operating rooms, where patients oftentimes receive multiple infusions simultaneously. This makes it difficult for clinicians to quickly and accurately identify the medication, infusion port location, and infusion pump parameters for each infusion line connected to a given patient.

Additionally, in healthcare environments involving a significant number of infusions, properly tracking infusion line expiration becomes increasingly difficult. Each infusion line has a limited lifespan, and it is important that clinicians replace infusion lines before they begin to degrade.

### SUMMARY

Current solutions for alleviating spaghetti syndrome, ensuring timely line replacement, or otherwise managing multiple infusion lines include organizers, labels, color indicators, lighting, time tags, and rudimentary reminders. However, none of these is particularly effective in removing the overall burden from the clinician. There is a need for innovative technologies that better address these and other issues faced by clinicians managing multiple infusion lines.

The present disclosure regards infusion sets with ultra-high frequency (UHF) radio-frequency identification (RFID) tags. These tags store important information associated with their respective infusion sets, such as treatment parameters, replacement timing, and patient information. A clinician can easily acquire this information by scanning the tags with an RFID reader, removing the need to dig through infusion lines, labels, or related paperwork.

Many UHF RFID tags can be read from and authenticated in a very short amount of time and at a notable distance. For example, solutions employing RAIN RFID tags may allow for simultaneous cryptographic authentication and data retrieval within one second's time from up to one thousand tags and at a distance of up to ten meters. Information stored at the UHF RFID tags can be relayed to a mobile device, whereat a clinician can view the information in a more convenient fashion.

Additionally, the present disclosure also regards infusion sets with locator devices affixed, for example, to the proximal end of their respective infusion lines (i.e., the end nearest the patient). Rather than requiring the clinician to trace a fluid container down to the end of the infusion set, the clinician can select an infusion set via an RFID reader or a mobile device—causing the locator device to light up, rumble, or otherwise alert the clinician regarding the location of the selected infusion set.

Benefits of the subject technology include potentially (i) eliminating the risk of vesicant medications administered via peripheral infusion lines, (ii) reducing delays and interruptions in administering critical medications to patients, (iii) decreasing the amount of time needed to identify the correct injection port for bolus administration, (iv) eliminating the need for line tracing (e.g., tracing an infusion line from a fluid bag attached thereto to the opposite end thereof), (v) eliminating the use of labels to avoid the risk of healthcare infection, (vi) eliminating medication incompatibility issues, (vii) avoiding administration of a discontinued medication or fluid, (viii) allowing for clinicians to access infusion pump parameters without the need to track in infusion line, (ix) enabling remote monitoring of an intravenous infusion system that includes multiple infusion lines, and/or (x) allowing for digital twins of corresponding infusion lines (and simplifying management of data associated therewith).

Example embodiments include at least the following:

A system includes a first infusion set and a second infusion set. The first infusion set includes an infusion line and a UHF RFID tag affixed to the infusion line. The UHF RFID tag is configured to receive information associated with the first infusion set, store the information, and transmit the information to an RFID reader. The second infusion set includes another infusion line and another UHF RFID tag affixed to the other infusion line. The other UHF RFID tag is configured to receive other information associated with the second infusion set, store the other information, and transmit the other information to the RFID reader. The RFID reader is configured to transmit to one or more servers the information and the other information after receiving the information and the other information from the UHF RFID tag and the other UHF RFID tag.

A system includes a UHF RFID tag and a locator device. The UHF RFID tag is configured to be affixed to an infusion line of a first infusion set. Additionally, the UHF RFID tag is also configured to receive information associated with the first infusion set, store the information, and transmit the information to an RFID reader. The RFID reader is configured to transmit the information to one or more servers after receiving the information from the UHF RFID tag. The locator device is configured to be affixed to the infusion line of the first infusion set. Additionally, the locator device is configured to receive an identification request from a mobile device and, responsive to receiving the identification request, illuminate an LED of the locator device, activate a speaker of the locator device, or activate a vibrator of the locator device.

A method of manufacturing infusion sets with UHF RFID tags includes providing a first infusion set including an infusion line. The method also includes providing a UHF RFID tag configured to receive information associated with the first infusion set, store the information, and transmit the information to an RFID reader. Additionally, the method includes affixing the UHF RFID tag to the infusion line. Further, the method includes providing a second infusion set including another infusion line. Moreover, the method includes providing another UHF RFID tag configured to receive other information associated with the second infusion set, store the other information, and transmit the other information to the RFID reader. Furthermore, the method includes affixing the other UHF RFID tag to the other infusion line. The RFID reader is configured to forward the information associated with the first infusion set and the other information associated with the second infusion set to one or more servers.

Other configurations of the subject technology will be apparent to those skilled in the art from the detailed description below, which describes various configurations of the subject technology and illustrations thereof. The subject technology is capable of other and different configurations, and its several details are capable of modification in various other respects, all without departing from the scope of the subject technology. Thus, the Drawings and Detailed Description are presented as illustrative in nature and should not be construed as restricting the present disclosure.

### BRIEF DESCRIPTION OF THE DRAWINGS

For a better understanding of the present disclosure, reference should be made to the Detailed Description, below, in conjunction with the following drawings. Like reference numerals refer to corresponding parts throughout the figures and the description.

FIG. 1 illustrates example infusion sets with respective UHF RFID tags and locator devices, according to various aspects of the subject technology.

FIG. 2 illustrates an example system for managing multiple infusion sets, according to various aspects of the subject technology.

FIGS. 3A and 3B illustrate an example mobile device for interfacing with multiple infusion sets, according to various aspects of the subject technology.

FIG. 4 illustrates an example process for streamlining management of multiple infusion sets, according to various aspects of the subject technology.

### DETAILED DESCRIPTION

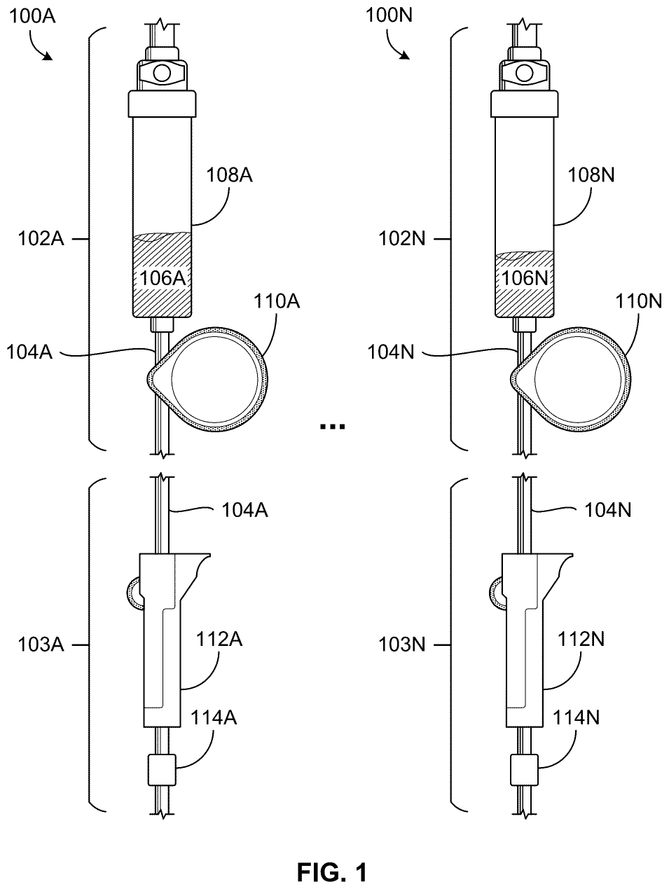

FIG. 1 illustrates example multiple infusion sets 100 (i.e., infusion sets 100A through 100N) with respective UHF RFID tags 110 and locator devices 114, according to various aspects of the subject technology. These infusion sets 100 can be used to treat a single patient in need of simultaneous administration multiple fluids 106 (e.g., medication, nutrition, hydration).

Each infusion set 100 is partitioned into distal and proximal portions 102-103. The distal portion 102 includes a drip chamber 108 for monitoring the flow rate of a fluid 106 in the infusion set 100, and the proximal portion 103 includes a roller clamp 112 for regulating the fluid flow rate. Additionally, an infusion line 104 runs between the distal and proximal portions 102-103, connecting the drip chamber 108 to a patient (e.g., via a catheter).

The infusion sets 100 also include respective UHF RFID tags 110 (e.g., a RAIN RFID tag), which store information associated with their respective sets 100. For example, the first UHF RFID tag 110A may store information including one or more of (i) a type of the infusion set 100A (e.g., primary, secondary), (ii) a type of the infusion line 104A (e.g., standard IV, PICC, CVC), (iii) an attribute of the fluid 106A (e.g., type, concentration, dose), (iv) personal information regarding the patient (e.g., name, date of birth), (v) identifying information regarding a clinician (e.g., name) responsible for the infusion set 100A, (vi) a parameter for administering the infusion therapy to the patient (e.g., flow rate, total volume, start time, end time), and (vii) a status of the infusion therapy (e.g., active, paused, completed). In the depicted embodiment, the UHF RFID tags 110 are each stored in the distal portions 102 of their respective infusion sets 100. However, the tags 110 can be stored anywhere on the infusion sets 100, including in the proximal portions 103 thereof.

The infusion sets 100 also include locator devices 114 for quickly locating the sets 100. These devices 114 may remove the need for clinicians to trace infusion lines 104 from fluid containers (e.g., located above drip chambers 108) down to the patient, which can be particularly difficult when the patient is receiving a significant number of infusions. The more infusion lines connected to a given patient, the more likely it is that the infusion lines will tangle or at least be difficult to navigate (i.e., “spaghetti syndrome”). The locator devices 114 can include, for example, light emitting diodes (LEDs), speakers, vibrators, and/or other components for calling the clinician's attention to the device 114. After receiving an identification request from the clinician (e.g., via a mobile device), the locator device 114 can then activate said components (e.g., illuminate an LED).

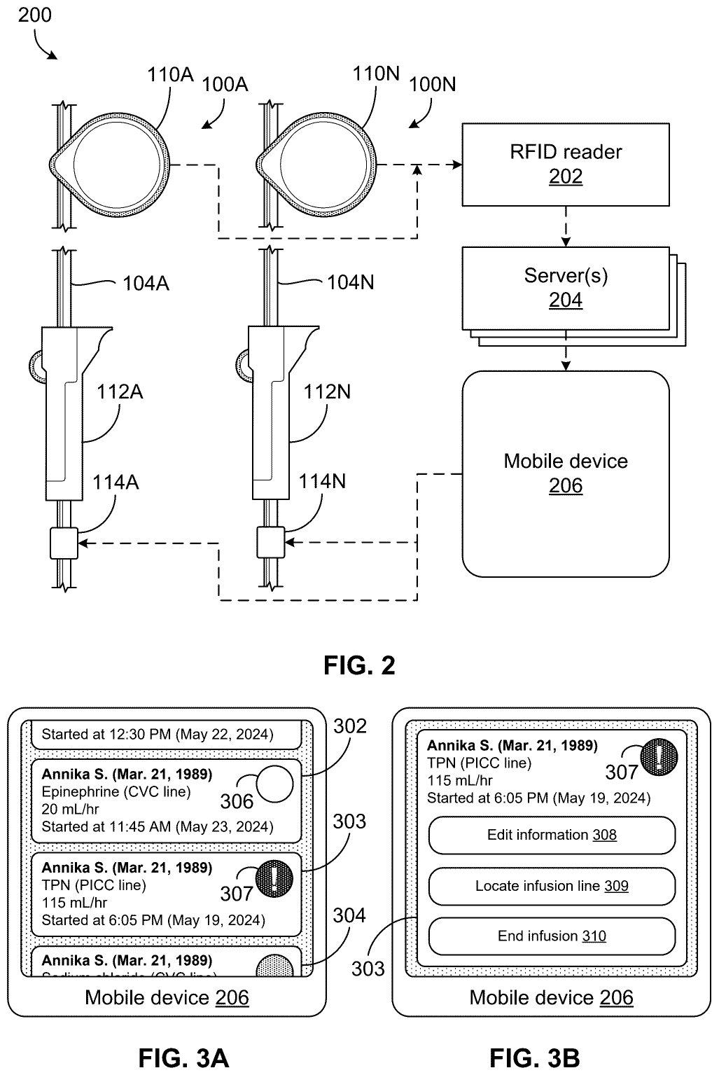

FIG. 2 illustrates an example system 200 for managing multiple infusion sets 100 (i.e., infusion sets 100A through 100N), according to various aspects of the subject technology. In addition to the infusion sets 100, the system 200 includes an RFID reader 202, one or more servers 204, and a mobile device 206.

The RFID reader 202 is configured to retrieve information from the UHF RFID tags 110, such as the aforenoted information associated with the infusion sets 100. Given the capabilities of UHF RFID technology, the clinician can retrieve information from each of the UHF RFID tags 110 with a single action. For instance, the clinician can bring the RFID reader within a certain distance of the UHF RFID tags 110 (e.g., 10 meters) and retrieve the information without needing to scan each of them individually.

After retrieving the information, the RFID reader 202 then transmits it to the one or more servers 204, which are configured to store the information for later retrieval by the mobile device 206. In some embodiments, the servers 204 include an internet-of-things (IoT) server or an electronic medical records (EMR) server, allowing for direct integration of the tags 110 into a larger IoT healthcare network or an EMR database. Additionally, in some embodiments, the servers 204 communicate with each UHF RFID tag 110 (e.g., via the RFID reader 202) and confirms that the tag 110 or corresponding information is authentic (e.g., using an authentication key) before storing the information. If the servers 204 fail to authenticate any of the UHF RFID tags 110, in some embodiments, the servers 204 notify the clinician regarding said failure.

After the one or more servers 204 store the information, the mobile device 206 then retrieves the information and displays it for review by the clinician (see FIGS. 3A and 3B). If the clinician determines that they need to review one of the infusion sets 100, the clinician can send an identification request from the mobile device 206 to the locator device 114 of the corresponding set 100. As discussed above, the locator devices 114 are configured to illuminate an LED or activate a speaker or vibrator to assist the clinician in locating the corresponding infusion set 100. For example, if the clinician sends an identification request to the first locator device 114A, the device 114A can illuminate an LED until the clinician terminates the identification request (e.g., via the mobile device 206). In some embodiments, the locator devices 114 are affixed to the infusion sets 100 in the proximal portion 103 thereof to assist the clinician in quickly locating the roller clamp 112 or the respective injection port.

In some embodiments, the information is automatically transferred from the RFID reader 202, to the one or more servers 204, and onto the mobile device 206. In this manner, the clinician may not need to perform any action beyond using the RFID reader 202 to scan the UHF RFID tags 110 to cause the information associated with the infusion sets 100 to appear at the mobile device 206, where the clinician can then view the information.

FIGS. 3A and 3B illustrate an example mobile device 206 for interfacing with multiple infusion sets (e.g., infusion sets 100A through 100N), according to various aspects of the subject technology. The mobile device 206 displays information retrieved from one or more servers (e.g., servers 204), including information regarding the name of the patient (e.g., “Annika S.”) associated with a particular infusion set, the type of the fluid being administered to the patient (e.g., “Epinephrine”) via the infusion set, and so on.

In FIG. 3A, the mobile device 206 displays information regarding multiple infusion sets, with the information broken up into individual blocks 302-304 each corresponding to a different set. In addition to the information each block 302-304 contains, the blocks 302-304 also include expiration indicators (e.g., expiration indicators 306-307) indicative of an expiration time of the infusion sets. For example, the second block 303 includes an expiration indicator 307 suggesting that the corresponding infusion set will expire soon (e.g., within 1 hour). By contrast, the expiration indicator 306 of the first block 306 suggests that the corresponding infusion set will not expire for a while (e.g., at least 24 hours). In this manner, the blocks 302-304 and expiration indicators 306-307 can assist a clinician in reviewing the various infusions for a given patient and determining whether the corresponding infusion sets need to be replaced.

In FIG. 3B, the mobile device 206 displays an expanded version of the second block 303, as well as its expiration indicator 307 and corresponding utility buttons 308-310. The first button 308 allows the clinician to edit information associated with the corresponding infusion set (e.g., infusion set 100A), whereafter the information can be transmitted to the corresponding UHF RFID tag (e.g., via one or more servers 204 and/or RFID reader 202). The second button 309 allows the clinician to send an identification request to the corresponding locator device (e.g., locator device 114A), which will cause the device to alert the clinician as discussed above. The third button 310 allows the clinician to end the infusion 310 for the corresponding infusion set. This may cause the mobile device 206 to stop displaying the second block 303 and/or transmit an identification request to the corresponding locator device so the clinician can easily disconnect the corresponding infusion set from the patient. Additionally, this may cause the mobile device to transmit updated information (e.g., via one or more servers 204 and/or RFID reader 202) to the corresponding UHF RFID tag.

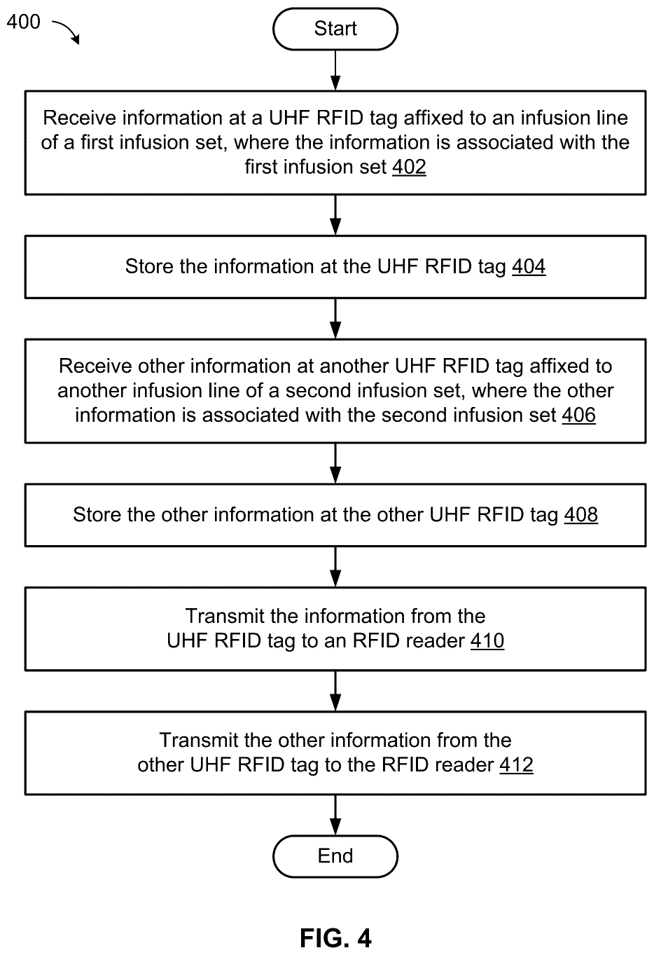

FIG. 4 illustrates an example process 400 for streamlining management of multiple infusion sets, according to various aspects of the subject technology. The operations of the process 400 can be executed at least in part by one or more of the devices discussed herein, such as the UHF RFID tags 110 and the locator devices 114, the RFID reader 202, the one or more servers 204, and the mobile device 206. In some embodiments, the process 300 is executed by a processor configured to execute instructions stored in a non-transitory, computer-readable medium, where said instructions correspond to the operations of the process 300.

The process 400 includes receiving (402) information at a UHF RFID tag (e.g., UHF RFID tag 110A) affixed to an infusion line (e.g., infusion line 104A) of a first infusion set (e.g., infusion set 100A) (or affixed elsewhere to the first infusion set), where the information is associated with the first infusion set. In some embodiments, the information is received from an RFID reader (e.g., RFID reader 202). The process 400 also includes storing (404) the information at the UHF RFID tag.

Additionally, the process 400 includes receiving (406) other information at another UHF RFID tag (e.g., UHF RFID tag 110N) affixed to another infusion line (e.g., infusion line 104N) of a second infusion set (e.g., infusion set 100N) (or affixed elsewhere to the second infusion set), where the other information is associated with the second infusion set. In some embodiments, the information is received from the aforenoted RFID reader. Further, the process 400 includes storing (408) the other information at the other UHF RFID tag.

Moreover, the process 400 includes transmitting (410) the information from the UHF RFID tag to an RFID reader (e.g., RFID reader 202) and transmitting (412) the other information from the other UHF RFID tag to the RFID reader. The RFID reader is configured to transmit to one or more servers (e.g., servers 204) the information and the other information after receiving the information and the other information from the UHF RFID tag and the other UHF RFID tag.

In some embodiments, the process 400 further includes receiving at a locator device (e.g., locator device 114A) an identification request from a mobile device (e.g., mobile device 206), where the locator device is affixed to the infusion line (or affixed elsewhere to the first infusion set). Additionally, the process 400 includes illuminating an LED of the locator device responsive to receiving the identification request. Further, the process 400 includes receiving at another locator device (e.g., locator device 114N) another identification request from the mobile device, where the other locator device is affixed to the other infusion line (or affixed elsewhere to the second infusion set). Moreover, the process 400 includes illuminating another LED of the other locator device responsive to receiving the other identification request.

In some embodiments, the process 400 includes activating a speaker or vibrator of the locator device responsive to receiving the identification request and activating another speaker or vibrator of the other locator device responsive to receiving the other identification request.

In some embodiments, the one or more servers are configured to, prior to receiving the information and the other information from the RFID reader, transmit an authentication request to the RFID reader including an authentication key (e.g., an encrypted code) and a patient identifier. Additionally, the RFID reader is configured to, prior to transmitting the information and the other information to the one or more servers: (i) receive the authentication request from the one or more servers, (ii) authenticate the one or more servers using the authentication key, and (iii) determine the information and the other information is associated with the patient identifier.

In some embodiments, a mobile device (e.g., mobile device 206) associated with a clinician responsible for the first and second infusion sets is configured to retrieve the information associated with the first infusion set and the other information associated with the second infusion set from the one or more servers after the RFID reader forwards the information and the other information to the one or more servers. Additionally, the mobile device is configured to display the information and the other information (see, e.g., FIGS. 3A and 3B).

In some embodiments, displaying the information and the other information includes displaying an expiration indicator (e.g., expiration indicator 306) indicative of an expiration time of the first infusion set and another expiration indicator (e.g., expiration indicator 307) indicative of another expiration time of the second infusion set.

In some embodiments, the expiration indicator is color coded to indicate the expiration time of the first infusion set and the other expiration indicator is color coded to indicate the other expiration time of the second infusion set. For example, the expiration indicators can be green to indicate that the infusion sets need not be replaced anytime soon (e.g., within 24 hours); yellow, to indicate that the infusion sets need to be replaced somewhat soon (e.g., within 12 hours); and red, to indicate that the infusion sets need to be replaced soon (e.g., within 1 hour) and/or should already have been replaced.

In some embodiments, the mobile device is further configured to determine that the first infusion set is nearing expiration based on the information or the second infusion set is nearing expiration based on the other information. Additionally, the mobile device is configured to notify the clinician that the first infusion set is nearing expiration responsive to detecting that the first infusion set is nearing expiration or the second infusion set is nearing expiration responsive to detecting that the second infusion set is nearing expiration.

In some embodiments, the information associated with the first infusion set includes one or more of (i) a type of the first infusion set, (ii) a type of the infusion line, (iii) an attribute of a fluid contained within a fluid container, wherein the infusion set further comprises the fluid container, (iv) personal information regarding a patient associated with the first infusion set, (v) identifying information regarding a clinician responsible for the first infusion set, (vi) a parameter for administration of an infusion therapy involving the first infusion set and the patient, and (vii) a status of the infusion therapy. Similarly, in some embodiments, the other information associated with the second infusion set includes one or more of (i) a type of the second infusion set, (ii) a type of the other infusion line, (iii) an attribute of another fluid contained within another fluid container, wherein the second infusion set further comprises the other fluid container, (iv) the personal information regarding the patient, wherein the patient is further associated with the second infusion set, (v) the identifying information regarding the clinician, wherein the clinician is further responsible for the second infusion set, (vi) a parameter for administration of another infusion therapy involving the second infusion set and the patient, and (vii) a status of the other infusion therapy.

Illustrative Clauses. For further reference, example aspects of the present disclosure are included below as numbered clauses. These clauses are provided for illustrative purposes and are not intended to limit the subject technology.

Clause 1. A system comprising: a first infusion set comprising: an infusion line; and a UHF RFID tag affixed to the infusion line and configured to: receive information associated with the first infusion set; store the information; and transmit the information to an RFID reader; and a second infusion set comprising: another infusion line; and another UHF RFID tag affixed to the other infusion line and configured to: receive other information associated with the second infusion set; store the other information; and transmit the other information to the RFID reader; wherein the RFID reader is configured to transmit to one or more servers the information and the other information after receiving the information and the other information from the UHF RFID tag and the other UHF RFID tag.

Clause 2. The system of Clause 1, further comprising: a locator device comprising an LED, wherein the locator device is affixed to the infusion line and configured to: receive an identification request from a mobile device; and illuminate the LED responsive to receiving the identification request; and another locator device comprising another LED, wherein the other locator device is affixed to the other infusion line and configured to: receive another identification request from the mobile device; and illuminate the other LED responsive to receiving the other identification request.

Clause 3. The system of Clause 2, wherein: the locator device further comprises a speaker or vibrator and is further configured to activate the speaker or vibrator responsive to receiving the identification request; and the other locator device further comprises another speaker or vibrator and is further configured to activate the other speaker or vibrator responsive to receiving the other identification request.

Clause 4. The system of Clause 1, further comprising: the RFID reader and the one or more servers; wherein the one or more servers are configured to, prior to receiving the information and the other information from the RFID reader, transmit an authentication request to the RFID reader comprising an authentication key and a patient identifier; and wherein the RFID reader is configured to, prior to transmitting the information and the other information to the one or more servers: receive the authentication request from the one or more servers; authenticate the one or more servers using the authentication key; and determine the information and the other information is associated with the patient identifier.

Clause 5. The system of Clause 1, further comprising: a mobile device associated with a clinician responsible for the first and second infusion sets, wherein the mobile device is configured to: retrieve the information associated with the first infusion set and the other information associated with the second infusion set from the one or more servers after the RFID reader forwards the information and the other information to the one or more servers; and display the information and the other information.

Clause 6. The system of Clause 5, wherein: displaying the information and the other information comprises displaying (i) an expiration indicator indicative of an expiration time of the first infusion set and (ii) another expiration indicator indicative of another expiration time of the second infusion set.

Clause 7. The system of Clause 5, wherein: the mobile device is further configured to: determine that (i) the first infusion set is nearing expiration based on the information or (ii) the second infusion set is nearing expiration based on the other information; and notify the clinician that (i) the first infusion set is nearing expiration responsive to detecting that the first infusion set is nearing expiration or (ii) the second infusion set is nearing expiration responsive to detecting that the second infusion set is nearing expiration.

Clause 8. The system of Clause 1, wherein: the information associated with the first infusion set comprises one or more of (i) a type of the first infusion set, (ii) a type of the infusion line, (iii) an attribute of a fluid contained within a fluid container, wherein the infusion set further comprises the fluid container, (iv) personal information regarding a patient associated with the first infusion set, (v) identifying information regarding a clinician responsible for the first infusion set, (vi) a parameter for administration of an infusion therapy involving the first infusion set and the patient, and (vii) a status of the infusion therapy; and the other information associated with the second infusion set comprises one or more of (i) a type of the second infusion set, (ii) a type of the other infusion line, (iii) an attribute of another fluid contained within another fluid container, wherein the second infusion set further comprises the other fluid container, (iv) the personal information regarding the patient, wherein the patient is further associated with the second infusion set, (v) the identifying information regarding the clinician, wherein the clinician is further responsible for the second infusion set, (vi) a parameter for administration of another infusion therapy involving the second infusion set and the patient, and (vii) a status of the other infusion therapy.

Clause 9. A system comprising: a UHF RFID tag configured to be affixed to an infusion line of a first infusion set and further configured to: receive information associated with the first infusion set; store the information; and transmit the information to an RFID reader configured to transmit the information to one or more servers after receiving the information from the UHF RFID tag; and a locator device configured to be affixed to the infusion line of the first infusion set and further configured to: receive an identification request from a mobile device; and responsive to receiving the identification request, (i) illuminate an LED of the locator device, (ii) activate a speaker of the locator device, or (iii) activate a vibrator of the locator device.

Clause 10. The system of Clause 9, wherein: wherein the one or more servers are configured to, prior to receiving the information from the RFID reader, transmit an authentication request to the RFID reader comprising an authentication key and a patient identifier; and wherein the RFID reader is configured to, prior to transmitting the information to the one or more servers, (i) receive the authentication request from the one or more servers, (ii) authenticate the one or more servers using the authentication key, and (iii) determine the information is associated with the patient identifier.

Clause 11. The system of Clause 9, wherein: after the information associated with the first infusion set is received at the one or more servers, the information is retrieved from the one or more servers by a mobile device associated with a clinician responsible for the first infusion set, wherein the mobile device is configured to retrieve the information from the one or more servers and display the information.

Clause 12. The system of Clause 11, wherein: displaying the information comprises displaying an expiration indicator indicative of an expiration time of the first infusion set.

Clause 13. The system of Clause 11, wherein: the mobile device is further configured to (i) determine that the first infusion set is nearing expiration based on the information and (ii) notify the clinician that the first infusion set is nearing expiration responsive to detecting that the first infusion set is nearing expiration.

Clause 14. The system of Clause 9, wherein: the information associated with the first infusion set comprises one or more of (i) a type of the first infusion set, (ii) a type of the infusion line, (iii) an attribute of a fluid contained within a fluid container, wherein the infusion set further comprises the fluid container, (iv) personal information regarding a patient associated with the first infusion set, (v) identifying information regarding a clinician responsible for the first infusion set, (vi) a parameter for administration of an infusion therapy involving the first infusion set and the patient, and (vii) a status of the infusion therapy.

Clause 15. The system of Clause 9, further comprising: another UHF RFID tag configured to be affixed to another infusion line of a second infusion set and further configured to: receive other information associated with the second infusion set; store the other information; and transmit the other information to the RFID reader, wherein the RFID reader is further configured to transmit the other information to the one or more servers after receiving the other information from the other UHF RFID tag; and another locator device configured to be affixed to the other infusion line of the second infusion set and further configured to: receive another identification request; and responsive to receiving the other identification request, (i) illuminate an LED of the other locator device, (ii) activate a speaker of the other locator device, or (iii) activate a vibrator of the other locator device.

Clause 16. The system of Clause 15, wherein: after the information associated with the first infusion set and the other information associated with the second infusion set are received at the one or more servers, the information and the other information are retrieved from the one or more servers by a mobile device associated with a clinician responsible for the first and second infusion sets, wherein the mobile device is configured to: retrieve the information and the other information from the one or more servers; display the information and the other information, wherein displaying the information and the other information comprises (i) displaying an expiration indicator indicative of an expiration time of the first infusion set and (ii) displaying another expiration indicator indicative of an expiration time of the second infusion set; determine (i) the first infusion set is nearing expiration based on the information or (ii) the second infusion set is nearing expiration based on the other information; and notify the clinician that (i) the first infusion set is nearing expiration responsive to detecting that the first infusion set is nearing expiration and (ii) the second infusion set is nearing expiration responsive to determining that the second infusion set is nearing expiration.

Clause 17. A method of manufacturing infusion sets with UHF RFID tags, comprising: providing a first infusion set comprising an infusion line; providing a UHF RFID tag configured to (i) receive information associated with the first infusion set, (ii) store the information, and (iii) transmit the information to an RFID reader; affixing the UHF RFID tag to the infusion line; providing a second infusion set comprising another infusion line; providing another UHF RFID tag configured to (i) receive other information associated with the second infusion set, (ii) store the other information, and (iii) transmit the other information to the RFID reader; affixing the other UHF RFID tag to the other infusion line; wherein the RFID reader is configured to forward the information associated with the first infusion set and the other information associated with the second infusion set to one or more servers.

Clause 18. The method of Clause 17, further comprising: providing a locator device comprising an LED and configured to (i) receive an identification request from a mobile device and (ii) illuminate the LED responsive to receiving the identification request; affixing the locator device to the infusion line; providing another locator device comprising another LED and configured to (i) receive another identification request from the mobile device and (ii) illuminate the other LED responsive to receiving the other identification request; and affixing the other locator device to the other infusion line.

Clause 19. The method of Clause 18, wherein: the locator device further comprises a speaker or vibrator and is further configured to activate the speaker or vibrator responsive to receiving the identification request; and the other locator device further comprises another speaker or vibrator and is further configured to activate the other speaker or vibrator responsive to receiving the other identification request.

Clause 20. The method of Clause 17, wherein: the information associated with the first infusion set comprises one or more of (i) a type of the first infusion set, (ii) a type of the infusion line, (iii) an attribute of a fluid contained within a fluid container, wherein the infusion set further comprises the fluid container, (iv) personal information regarding a patient associated with the first infusion set, (v) identifying information regarding a clinician responsible for the first infusion set, and (vi) a parameter for administration of an infusion therapy involving the first infusion set and the patient; and the other information associated with the second infusion set comprises one or more of (i) a type of the second infusion set, (ii) a type of the other infusion line, (iii) an attribute of another fluid contained within another fluid container, wherein the second infusion set further comprises the other fluid container, (iv) the personal information regarding the patient, wherein the patient is further associated with the second infusion set, (v) the identifying information regarding the clinician, wherein the clinician is further responsible for the second infusion set, and (vi) a parameter for administration of another infusion therapy involving the second infusion set and the patient.

Further Consideration. The specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims pre-sent elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not in-tended to be limited to the aspects shown herein but are to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the invention described herein.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” and “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be appreciated that a message may be composed, transmitted, stored, received, and so on in multiple parts.

As used herein, the term “selectively” or “selective” may encompass a wide variety of actions. For example, a “selective” process may include determining one option from multiple options. A “selective” process may include one or more of: dynamically determined in-puts, preconfigured inputs, or user-initiated inputs for making the determination. In some embodiments, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine-learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A system comprising:
a first infusion set comprising:
an infusion line; and
a UHF RFID tag affixed to the infusion line and configured to:
receive information associated with the first infusion set;
store the information; and
transmit the information to an RFID reader; and
a second infusion set comprising:
another infusion line; and
another UHF RFID tag affixed to the other infusion line and configured to:
receive other information associated with the second infusion set;
store the other information; and
transmit the other information to the RFID reader;
wherein the RFID reader is configured to transmit to one or more servers the information and the other information after receiving the information and the other information from the UHF RFID tag and the other UHF RFID tag.

2. The system of claim 1, further comprising:
a locator device comprising an LED, wherein the locator device is affixed to the infusion line and configured to:
receive an identification request from a mobile device; and
illuminate the LED responsive to receiving the identification request; and
another locator device comprising another LED, wherein the other locator device is affixed to the other infusion line and configured to:
receive another identification request from the mobile device; and
illuminate the other LED responsive to receiving the other identification request.

3. The system of claim 2, wherein:
the locator device further comprises a speaker or vibrator and is further configured to activate the speaker or vibrator responsive to receiving the identification request; and
the other locator device further comprises another speaker or vibrator and is further configured to activate the other speaker or vibrator responsive to receiving the other identification request.

4. The system of claim 1, further comprising:
the RFID reader and the one or more servers;
wherein the one or more servers are configured to, prior to receiving the information and the other information from the RFID reader, transmit an authentication request to the RFID reader comprising an authentication key and a patient identifier; and
wherein the RFID reader is configured to, prior to transmitting the information and the other information to the one or more servers:
receive the authentication request from the one or more servers;
authenticate the one or more servers using the authentication key; and
determine the information and the other information is associated with the patient identifier.

5. The system of claim 1, further comprising:
a mobile device associated with a clinician responsible for the first and second infusion sets, wherein the mobile device is configured to:
retrieve the information associated with the first infusion set and the other information associated with the second infusion set from the one or more servers after the RFID reader forwards the information and the other information to the one or more servers; and
display the information and the other information.

6. The system of claim 5, wherein:
displaying the information and the other information comprises displaying (i) an expiration indicator indicative of an expiration time of the first infusion set and (ii) another expiration indicator indicative of another expiration time of the second infusion set.

7. The system of claim 5, wherein:
the mobile device is further configured to:
determine that (i) the first infusion set is nearing expiration based on the information or (ii) the second infusion set is nearing expiration based on the other information; and
notify the clinician that (i) the first infusion set is nearing expiration responsive to detecting that the first infusion set is nearing expiration or (ii) the second infusion set is nearing expiration responsive to detecting that the second infusion set is nearing expiration.

8. The system of claim 1, wherein:
the information associated with the first infusion set comprises one or more of (i) a type of the first infusion set, (ii) a type of the infusion line, (iii) an attribute of a fluid contained within a fluid container, wherein the infusion set further comprises the fluid container, (iv) personal information regarding a patient associated with the first infusion set, (v) identifying information regarding a clinician responsible for the first infusion set, (vi) a parameter for administration of an infusion therapy involving the first infusion set and the patient, and (vii) a status of the infusion therapy; and
the other information associated with the second infusion set comprises one or more of (i) a type of the second infusion set, (ii) a type of the other infusion line, (iii) an attribute of another fluid contained within another fluid container, wherein the second infusion set further comprises the other fluid container, (iv) the personal information regarding the patient, wherein the patient is further associated with the second infusion set, (v) the identifying information regarding the clinician, wherein the clinician is further responsible for the second infusion set, (vi) a parameter for administration of another infusion therapy involving the second infusion set and the patient, and (vii) a status of the other infusion therapy.

9. A system comprising:
a UHF RFID tag configured to be affixed to an infusion line of a first infusion set and further configured to:
receive information associated with the first infusion set;
store the information; and
transmit the information to an RFID reader configured to transmit the information to one or more servers after receiving the information from the UHF RFID tag; and
a locator device configured to be affixed to the infusion line of the first infusion set and further configured to:
receive an identification request from a mobile device; and
responsive to receiving the identification request, (i) illuminate an LED of the locator device, (ii) activate a speaker of the locator device, or (iii) activate a vibrator of the locator device.

10. The system of claim 9, wherein:
wherein the one or more servers are configured to, prior to receiving the information from the RFID reader, transmit an authentication request to the RFID reader comprising an authentication key and a patient identifier; and
wherein the RFID reader is configured to, prior to transmitting the information to the one or more servers, (i) receive the authentication request from the one or more servers, (ii) authenticate the one or more servers using the authentication key, and (iii) determine the information is associated with the patient identifier.

11. The system of claim 9, wherein:
after the information associated with the first infusion set is received at the one or more servers, the information is retrieved from the one or more servers by a mobile device associated with a clinician responsible for the first infusion set, wherein the mobile device is configured to retrieve the information from the one or more servers and display the information.

12. The system of claim 11, wherein:
displaying the information comprises displaying an expiration indicator indicative of an expiration time of the first infusion set.

13. The system of claim 11, wherein:
the mobile device is further configured to (i) determine that the first infusion set is nearing expiration based on the information and (ii) notify the clinician that the first infusion set is nearing expiration responsive to detecting that the first infusion set is nearing expiration.

14. The system of claim 9, wherein:
the information associated with the first infusion set comprises one or more of (i) a type of the first infusion set, (ii) a type of the infusion line, (iii) an attribute of a fluid contained within a fluid container, wherein the infusion set further comprises the fluid container, (iv) personal information regarding a patient associated with the first infusion set, (v) identifying information regarding a clinician responsible for the first infusion set, (vi) a parameter for administration of an infusion therapy involving the first infusion set and the patient, and (vii) a status of the infusion therapy.

15. The system of claim 9, further comprising:
another UHF RFID tag configured to be affixed to another infusion line of a second infusion set and further configured to:
receive other information associated with the second infusion set;
store the other information; and
transmit the other information to the RFID reader, wherein the RFID reader is further configured to transmit the other information to the one or more servers after receiving the other information from the other UHF RFID tag; and
another locator device configured to be affixed to the other infusion line of the second infusion set and further configured to:
receive another identification request; and
responsive to receiving the other identification request, (i) illuminate an LED of the other locator device, (ii) activate a speaker of the other locator device, or (iii) activate a vibrator of the other locator device.

16. The system of claim 15, wherein:
after the information associated with the first infusion set and the other information associated with the second infusion set are received at the one or more servers, the information and the other information are retrieved from the one or more servers by a mobile device associated with a clinician responsible for the first and second infusion sets, wherein the mobile device is configured to:
retrieve the information and the other information from the one or more servers;
display the information and the other information, wherein displaying the information and the other information comprises (i) displaying an expiration indicator indicative of an expiration time of the first infusion set and (ii) displaying another expiration indicator indicative of an expiration time of the second infusion set;
determine (i) the first infusion set is nearing expiration based on the information or (ii) the second infusion set is nearing expiration based on the other information; and
notify the clinician that (i) the first infusion set is nearing expiration responsive to detecting that the first infusion set is nearing expiration and (ii) the second infusion set is nearing expiration responsive to determining that the second infusion set is nearing expiration.

17. A method of manufacturing infusion sets with UHF RFID tags, comprising:
providing a first infusion set comprising an infusion line;
providing a UHF RFID tag configured to (i) receive information associated with the first infusion set, (ii) store the information, and (iii) transmit the information to an RFID reader;
affixing the UHF RFID tag to the infusion line;
providing a second infusion set comprising another infusion line;
providing another UHF RFID tag configured to (i) receive other information associated with the second infusion set, (ii) store the other information, and (iii) transmit the other information to the RFID reader;
affixing the other UHF RFID tag to the other infusion line;
wherein the RFID reader is configured to forward the information associated with the first infusion set and the other information associated with the second infusion set to one or more servers.

18. The method of claim 17, further comprising:
providing a locator device comprising an LED and configured to (i) receive an identification request from a mobile device and (ii) illuminate the LED responsive to receiving the identification request;
affixing the locator device to the infusion line;
providing another locator device comprising another LED and configured to (i) receive another identification request from the mobile device and (ii) illuminate the other LED responsive to receiving the other identification request; and
affixing the other locator device to the other infusion line.

19. The method of claim 18, wherein:
the locator device further comprises a speaker or vibrator and is further configured to activate the speaker or vibrator responsive to receiving the identification request; and
the other locator device further comprises another speaker or vibrator and is further configured to activate the other speaker or vibrator responsive to receiving the other identification request.

20. The method of claim 17, wherein:
the information associated with the first infusion set comprises one or more of (i) a type of the first infusion set, (ii) a type of the infusion line, (iii) an attribute of a fluid contained within a fluid container, wherein the infusion set further comprises the fluid container, (iv) personal information regarding a patient associated with the first infusion set, (v) identifying information regarding a clinician responsible for the first infusion set, and (vi) a parameter for administration of an infusion therapy involving the first infusion set and the patient; and
the other information associated with the second infusion set comprises one or more of (i) a type of the second infusion set, (ii) a type of the other infusion line, (iii) an attribute of another fluid contained within another fluid container, wherein the second infusion set further comprises the other fluid container, (iv) the personal information regarding the patient, wherein the patient is further associated with the second infusion set, (v) the identifying information regarding the clinician, wherein the clinician is further responsible for the second infusion set, and (vi) a parameter for administration of another infusion therapy involving the second infusion set and the patient.
